(19) United States
(12) Patent Application Publication
(10) Pub. No.: US 2014/0073890 A1
(43) Pub. Date: Mar. 13, 2014

(54) SYSTEMS AND METHODS FOR DETERMINING FLUID RESPONSIVENESS

(71) Applicants: Mark Su, Boulder, CO (US); Bo Chen, Louisville, CO (US)

(72) Inventors: Mark Su, Boulder, CO (US); Bo Chen, Louisville, CO (US)

(73) Assignee: Nellcor Puritan Bennett LLC, Boulder, CO (US)

(21) Appl. No.: 13/611,269

(22) Filed: Sep. 12, 2012

(51) Int. Cl. A61B 5/0205; A61B 5/1455

(52) U.S. Cl. 600/324; 600/484

(57) ABSTRACT

A system is provided including a respiratory detection module, a circulatory detection module, and an analysis module. The respiratory detection module is configured to detect respiratory information representative of respiration of a patient. The circulatory detection module configured to detect circulatory information representative of circulation of the patient. The analysis module is configured to obtain a respiratory waveform based at least in part on the respiratory information, obtain a circulatory waveform based at least in part on the circulatory information, combine the respiratory waveform and the circulatory waveform to provide a mixed waveform, and isolate a portion of the mixed waveform to identify a respiratory responsiveness waveform representative of an effect of the respiration of the patient on the mixed waveform.

### FIELD

Embodiments of the present disclosure generally relate to physiological signal processing, and more particularly, to processing signals to determine the fluid responsiveness of a patient.

### BACKGROUND

A physician or nurse may use an index of fluid responsiveness to help determine whether the blood flow of a patient will benefit from additional fluid administration. The indices are typically used in connection with ventilated patients. Such dynamic preload indices may be based on a ventilator-induced variation of an arterial-line pressure waveform or a photoplethysmographic (“PPG”) waveform. The waveform variation may be caused by the following: 1) a breathing or respiratory cycle induces a cyclic increase in intrathoracic pressure, which causes 2) a cyclic reduction in venous return, which in turn causes 3) a cyclic reduction in preload, which causes 4) a cyclic reduction in cardiac output, which is manifested as 5) a cyclic variation in the arterial line pressure or PPG waveform. A large waveform variation indicates that cardiac output can probably be increased with fluid administration.

However, dynamic indices based on waveform variation are fluid-response predictive only at relative extremes of large waveform variation induced by high-tidal-volume ventilation. The use of lung-protective ventilation strategies for patients with acute lung injury (ALI) or acute respiratory distress syndrome (ARDS) means that many of the most critical patients do not have a large enough ventilation-induced waveform variation to use as a fluid-responsiveness measure with certain known techniques. Further still, the interpretation of dynamic indices or measurements used to arrive at such interpretations may be confounded by a number of factors. For example, artifacts introduced into the signal by sources other than the ventilator-induced changes in intrathoracic pressure may confound the analysis. As another example, differences in ventilator mode, circuit impedance, pressure and flow settings can all affect the size of the ventilator-induced waveform variability. Yet further still, because the indices are typically used in connection with ventilated patients, determinations regarding whether non-ventilated patients would benefit from fluid administration are made without the benefit of such indices. A need exists for improved determination of fluid responsiveness.

### SUMMARY

Certain embodiments of the present disclosure provide a system that may include a respiratory detection module, a circulatory detection module, and an analysis module. The respiratory detection module is configured to detect respiratory information representative of respiration of a patient. The circulatory detection module is configured to detect circulatory information representative of circulation of the patient. The analysis module is configured to obtain a respiratory waveform based at least in part on the respiratory information, obtain a circulatory waveform based at least in part on the circulatory information, combine the respiratory waveform and the circulatory waveform to provide a mixed waveform, and isolate a portion of the mixed waveform to identify a respiratory responsiveness waveform representative of an effect of the respiration of the patient on the mixed waveform.

The analysis module may be further configured to determine a fluid responsiveness parameter representative of fluid responsiveness of the patient using the respiratory responsiveness waveform.

The analysis module may be further configured to combine the respiratory waveform and the circulatory waveform by multiplication.

In some embodiments, the circulatory detection module may include a pulse oximetry sensor configured to provide photoplethysmographic information representative of a photopleythsmographic waveform of the ventilated patient. In some embodiments, the circulatory detection module may include an arterial line catheter and a pressure transducer. The pressure transducer is configured to be associated with the arterial line catheter and to provide blood pressure information representative of a blood pressure waveform of the ventilated patient.

The system may be configured to be operably connected to a non-ventilated patient. In some embodiments, the fluid responsiveness parameter may be determined with or without the patient being operably connected to a ventilator.

The respiratory detection module may include a CO₂ sensor, and the respiratory information may correspond to a level of CO₂ in exhaled breath.

Certain embodiments provide a method for determining fluid responsiveness. The method includes obtaining a respiratory waveform representative of respiratory output of a patient. The respiratory waveform is based on information obtained from a respiratory detection module. The method also includes obtaining a circulatory waveform representative of the circulation of the patient. The circulatory waveform is based on information provided by a circulatory detection module. The method further includes combining, at a processing module, the respiratory waveform and the circulatory waveform to provide a mixed waveform. Further, the method includes isolating, at a processing module, a portion of the mixed waveform to provide a respiratory responsiveness waveform representative of an effect of respiration on the mixed waveform.

Certain embodiments provide a tangible and non-transitory computer readable medium including one or more computer software modules. The one or more computer software modules are configured to direct a processor to obtain a respiratory waveform representative of a respiratory output of a patient. The respiratory waveform is based on information obtained from a respiratory detection module. Also, the one or more computer software modules are configured to direct a processor to obtain a circulatory waveform representative of the circulation of the ventilated patient. The circulatory waveform is based on information provided by a circulatory detection module. Further, the one or more computer software modules are configured to direct a processor to combine the respiratory waveform and the circulatory waveform to provide a mixed waveform, and isolate a portion of the mixed waveform to provide a respiratory responsiveness waveform representative of an effect of respiration on the mixed waveform.

Embodiments provide for the isolation of respiration variability (e.g. variation caused by respiration) in a waveform from other variability (e.g. variation caused by one or more other sources of potential variability), thereby allowing for a more controlled study and determination of fluid responsiveness. For example, embodiments provide systems and methods that are configured to more accurately determine a fluid responsiveness index or indices. Also, embodiments provide improved predictive value of fluid responsiveness determinations. Further, embodiments provide systems and methods that are configured to allow a determination of fluid responsiveness at relatively low tidal volume ventilation. Further still, embodiments provide systems and methods configured to determine a fluid responsiveness index for non-ventilated patients. Also, embodiments provide systems and methods configured to determine of fluid responsiveness for smaller variations of waveforms.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

FIG. 1 illustrates a schematic diagram, of a system for determining fluid responsiveness according to an embodiment.

FIG. 2 illustrates an isometric view of a photoplethysmogram (PPG) system according to an embodiment.

FIG. 3 illustrates a simplified block diagram of a PPG system in according to an embodiment.

FIG. 4 illustrate a PPG signal according to an embodiment.

FIG. 5 illustrates an isometric view of a monitoring system according to an embodiment.

FIG. 6 illustrates a flowchart of a method for determining fluid responsiveness according to an embodiment.

FIG. 7 illustrates a depiction of signal variability according to an embodiment.

FIG. 8 illustrates a flowchart of a method for determining fluid responsiveness according to an embodiment.

FIGS. 9a and 9b illustrate a mixed waveform according to an embodiment.

### DETAILED DESCRIPTION

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Embodiments of the present disclosure provide for the isolation of respiration variability (e.g. variation caused by respiration) in a waveform from other variability (e.g. variation caused by one or more other sources of potential variability), thereby allowing for a more controlled study and determination of fluid responsiveness. For example, embodiments provide systems and methods that are configured to more accurately determine a fluid responsiveness index or indices. Further still, embodiments provide systems and methods configured to determine a fluid responsiveness index for non-ventilated patients.

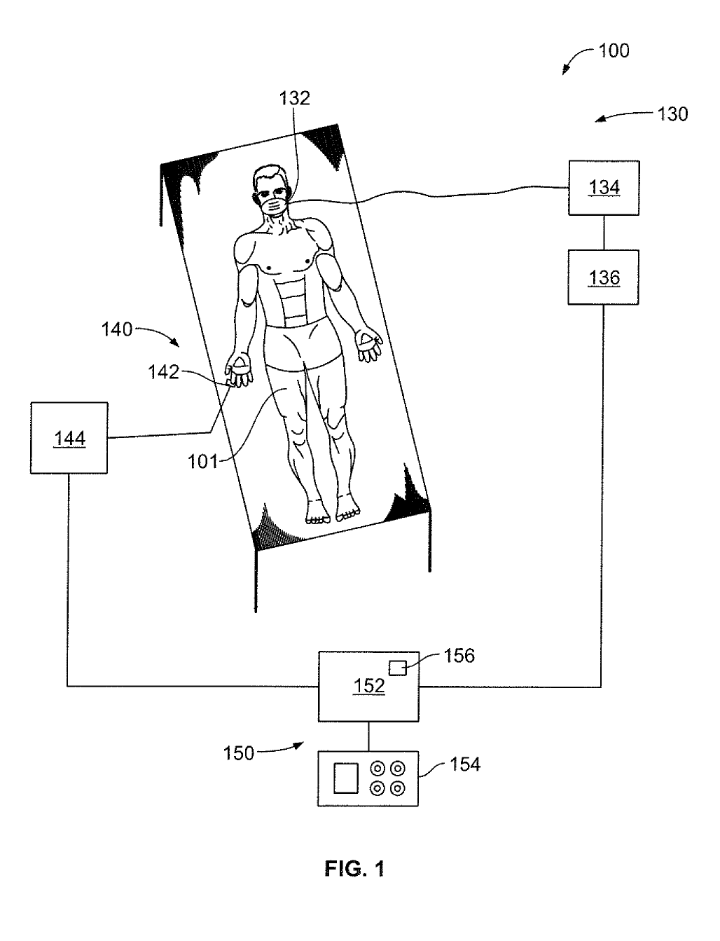

FIG. 1 illustrates a schematic diagram of a system 100 for determining fluid responsiveness in accordance with various embodiments. The system 100, for example, may be used in conjunction with embodiments or aspects of methods described elsewhere herein. The system 100 includes a respiratory detection module 130, a circulatory detection module 140, and a fluid responsiveness analysis module 150. In the illustrated embodiment, the system 100 includes two physiological detection modules, namely, the respiratory detection module 130 and the circulatory detection module 140. In alternate embodiments, different numbers and/or types of physiological detection modules may be employed. In the illustrated embodiment, the fluid responsiveness analysis module 150 is configured to determine fluid responsiveness (e.g. a parameter such as an index representative of the fluid responsiveness of the patient 101) using information provided by the respiratory detection module 130, and the circulatory detection module 140.

The various systems, modules, and units disclosed herein may include a controller, such as a computer processor or other logic-based device that performs operations based on one or more sets of instructions (e.g., software). The instructions on which the controller operates may be stored on a tangible and non-transitory (e.g., not a transient signal) computer readable storage medium, such as a memory. The memory may include one or more computer hard drives, flash drives, RAM, ROM, EEPROM, and the like. Alternatively, one or more of the sets of instructions that direct operations of the controller may be hard-wired into the logic of the controller, such as by being hard-wired logic formed in the hardware of the controller.

In the embodiment illustrated in FIG. 1, a patient 101 is shown being monitored by the system 100. The respiratory detection module 130 is configured to sense one or more outputs or characteristics of the respiration of the patient 101, and to provide information representative of the sensed characteristics to the fluid responsiveness analysis module 150. For example, in the illustrated embodiment, the respiratory detection module 130 includes a collection unit 132, a respiratory detector 134 and a respiratory detector processing unit 136. The respiratory collection unit 132 is configured to collect samples of the breath of the patient 101. In the illustrated embodiment, the respiratory collection unit 132 includes a mask. In alternate embodiments, the respiratory collection unit 132 may include a cannula positioned proximate to a patient's nostrils. In still further alternate embodiments, for example, embodiments used in conjunction with ventilated patients, the respiratory collection unit 132 may be associated with a tube or breathing circuit of a ventilation system. In the illustrated embodiment, the respiratory collection unit 132 is operably connected to the respiratory detector 134 via a pump (not shown) that draws breath samples from the respiratory collection unit 132 to the respiratory detector 134.

The respiratory detector 134 is configured to detect a property or output of the respiration of the patient 101, and to provide information representative of the detected property or output to the respiratory detector processing unit 136. The respiratory detector 134 may include appropriate sensors or sensor elements for assessing or determining expired carbon dioxide. In various embodiments, chemical, electrical, optical, non-optical, quantum-restricted, electrochemical, enzymatic, spectrophotometric, fluorescent, or chemiluminescent indicators or transducers may be employed.

The respiratory detector processing unit 136 then constructs and processes (e.g. by filtering or normalizing) a waveform using information provided by the respiratory detector 134, and in turn provides the waveform to the fluid responsiveness analysis module 150. Further still, the respiratory detector processing unit 136 may include a display and/or user interface allowing adjustment or selection of modes of processing of a respiratory waveform constructed using information provided by the respiratory detector 134. In other embodiments, the respiratory detector 134 may provide the information directly to the fluid responsiveness analysis module 150, with some or all of the functionality of the respiratory detector processing unit 136 incorporated into the fluid responsiveness analysis module 150.

The circulatory detection module 140 is configured to sense one or more circulatory characteristics of the patient 101, and to provide information representative of the sensed characteristics to the fluid responsiveness analysis module 150. For example, the circulatory detection module 140 in some embodiments is configured to detect a PPG or, as another example, an arterial line pressure. In the illustrated embodiment, the circulatory detection module 140 includes a circulatory detector 142 and a circulatory detector processing unit 144. The circulatory detector 142 is configured to detect a circulatory property or characteristic of the patient 101, and to provide information representative of the detected property or characteristic to the circulatory detector processing unit 144. For example, in the illustrated embodiment, the circulatory detector 142 includes a pulse oximeter configured for placement proximal to a finger of the patient 101 as depicted in the illustrated embodiment. The circulatory detector processing unit 144 then constructs and processes (e.g. filtering or normalizing) a waveform using information provided by the circulatory detector 142, and in turn provides the waveform to the fluid responsiveness analysis module 150. Further still, the circulatory detector processing unit 144 may include a display and/or user interface allowing adjustment or selection of modes of processing of a circulatory waveform constructed using information provided by the circulatory detector 142. In other embodiments, the circulatory detector 142 may provide the information directly to the fluid responsiveness analysis module 150, with some or all of the functionality of the circulatory detector processing unit 144 incorporated into the fluid responsiveness analysis module 150.

The fluid responsiveness analysis module 150 is configured to receive information from the respiratory detection module 130 as well as the physiological detection module 140, and to determine a measure or indication of fluid responsiveness using the provided information. The information may be provided in the form of one or more waveforms and/or one or more datasets that may be used to construct a waveform. For example, the fluid responsiveness analysis module 150 may receive respiratory information from the respiratory detection module 130 and construct a respiratory waveform using the respiratory information. The fluid responsiveness analysis module 150 may also receive circulatory information (e.g. PPG information) from the circulatory detection module 140 and construct a circulatory waveform using the circulatory information. In other embodiments, the fluid responsiveness analysis module 150 may receive one or more waveforms constructed by one or more of the respective detection modules. Further still, the fluid responsiveness analysis module 150, in some embodiments, is configured to process received information and/or waveforms, for example by filtering to remove noise or other artifacts, or, as another example, to synchronize two waveforms to each other.

The fluid responsiveness analysis module 150 is further configured to isolate information representing variability due to respiration from information representing variability due to other sources. For example, in some embodiments, the fluid responsiveness analysis module 150 is configured to apply a lock-in detection technique. The lock-in detection technique may be accomplished by synchronizing the respiratory waveform and the circulatory waveform, multiplying the two waveforms to provide a mixed waveform, and then applying a low pass filter to the mixed waveform to provide a respiratory responsiveness waveform. The variability of the respiratory responsiveness waveform provides an indication of the effect of respiration partially or entirely separated from other sources of potential variation in the mixed waveform. The respirator responsiveness waveform may then be analyzed by the fluid responsiveness analysis module 150, or additionally or alternatively by a practitioner, to determine fluid responsiveness, for example a fluid responsiveness variability index. For example, the variability of the respiratory responsiveness waveform may be analyzed to provide an index that may be correlated by clinical studies to a threshold for determining whether additional fluid administration is appropriate.

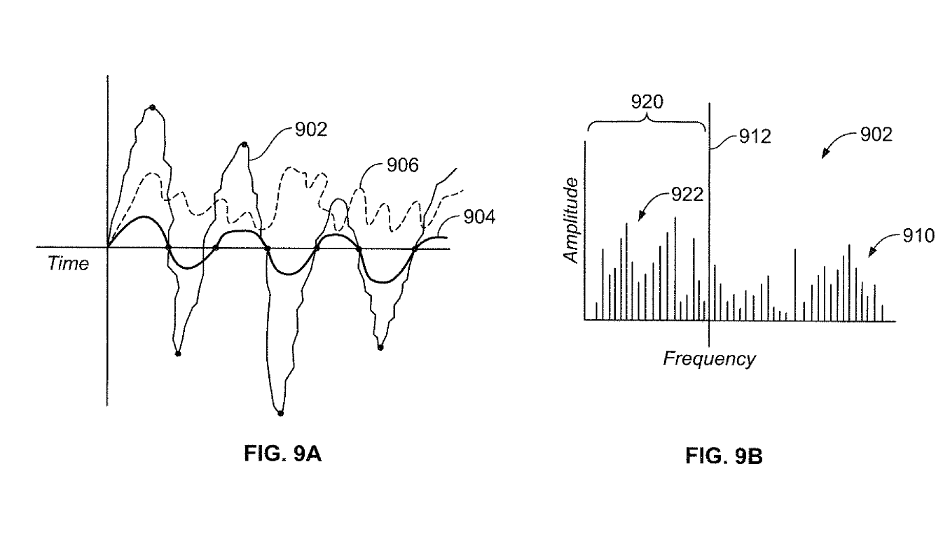

In the illustrated embodiment, the fluid responsiveness analysis module 150 is depicted as a stand-alone unit including a processing module 152 and a display module 154. The processing module 152, for example, may be configured to receive first and second physiological waveforms (e.g. a respiratory waveform and a circulatory waveform), multiply the two waveforms to obtain a mixed waveform, apply a low-pass filter to the mixed waveform to obtain a fluid responsiveness waveform, and determine a fluid responsiveness parameter using the fluid responsiveness waveform. (See, e.g. FIGS. 9a and 9b and related discussion.) In the illustrated embodiment, the fluid responsiveness analysis module 150 includes a lock-in detection module 156 configured to multiply the composite waveform and the physiological waveform and apply a low-pass filter. For example, the lock-in detection module 156 may include a lock-in amplifier.

The processing module 152 may, in some embodiments, be further configured to determine a fluid administration recommendation using the fluid responsiveness parameter. The display module 154, for example, may include a graphic user interface that displays a computed measure of respiratory responsiveness variability, such as an index, and/or displays a recommendation regarding whether additional fluid administration is appropriate. The graphic user interface of the display module 154 may also be configured to allow a practitioner to adjust settings of the fluid responsiveness analysis module 150. In still other embodiments, the fluid responsiveness analysis module 150 may be incorporated into a monitor or processing unit that also provides additional functionality. For example, in some embodiments, the fluid responsiveness analysis module 150 may be incorporated into a multi-parameter monitoring system.

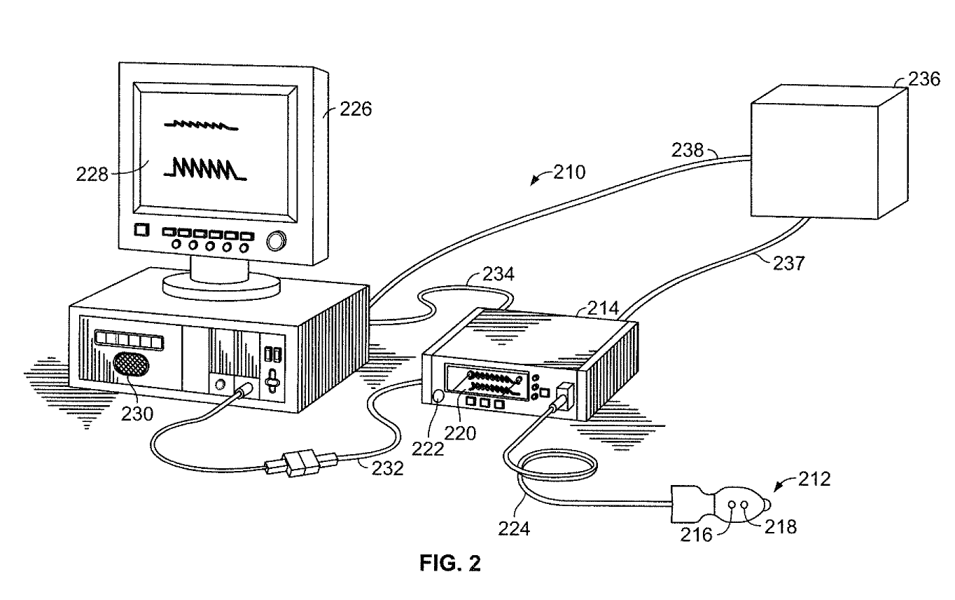

FIG. 2 illustrates an isometric view of a physiological detection system 210. The physiological detection system 210 includes an example of a circulatory detection module 140 as shown and described with respect to FIG. 1. For example, in the illustrated embodiment, the physiological detection system is configured as a PPG system 210. While the physiological system is shown and described as a PPG system 210, the system may be various other types of physiological detection systems, such as an arterial pressure detecting system including, for example, an arterial line catheter. The PPG system 210 may be a pulse oximetry system, for example. The PPG system 210 may include a PPG sensor 212 and a PPG monitor 214. The PPG sensor 212 may include an emitter 216 configured to emit light into tissue of a patient. For example, the emitter 216 may be configured to emit light at two or more wavelengths into the tissue of the patient. The PPG sensor 212 may also include a detector 218 that is configured to detect the emitted light from the emitter 216 that emanates from the tissue after passing through the tissue.

The PPG system 210 may include a plurality of sensors forming a sensor array in place of the PPG sensor 212. Each of the sensors of the sensor array may be a complementary metal oxide semiconductor (CMOS) sensor, for example. Alternatively, each sensor of the array may be a charged coupled device (CCD) sensor. In another embodiment, the sensor array may include a combination of CMOS and CCD sensors. The CCD sensor may include a photoactive region and a transmission region configured to receive and transmit, while the CMOS sensor may include an integrated circuit having an array of pixel sensors. Each pixel may include a photodetector and an active amplifier.

The emitter 216 and the detector 218 may be configured to be located at opposite sides of a digit, such as a finger or toe, in which case the light that is emanating from the tissue passes completely through the digit. The emitter 216 and the detector 218 may be arranged so that light from the emitter 216 penetrates the tissue and is reflected by the tissue into the detector 218, such as a sensor designed to obtain pulse oximetry data.

The sensor 212 or sensor array may be operatively connected to and draw power from the monitor 214. Optionally, the sensor 212 may be wirelessly connected to the monitor 214 and include a battery or similar power supply (not shown). The monitor 214 may be configured to calculate physiological parameters based at least in part on data received from the sensor 212 relating to light emission and detection. Alternatively, the calculations may be performed by and within the sensor 212 and the result of the oximetry reading may be passed to the monitor 214. Additionally, the monitor 214 may include a display 220 configured to display the physiological parameters or other information about the PPG system 210. The monitor 214 may also include a speaker 222 configured to provide an audible sound that may be used in various other embodiments, such as for example, sounding an audible alarm in the event that physiological parameters are outside a predefined normal range.

The sensor 212, or the sensor array, may be communicatively coupled to the monitor 214 via a cable 224. Alternatively, a wireless transmission device (not shown) or the like may be used instead of, or in addition to, the cable 224.

The PPG system 210 may also include a multi-parameter workstation 226 operatively connected to the monitor 214. The workstation 226 may be or include a computing sub-system 230, such as standard computer hardware. The computing sub-system 230 may include one or more modules and control units, such as processing devices that may include one or more microprocessors, microcontrollers, integrated circuits, memory, such as read-only and/or random access memory, and the like. The workstation 226 may include a display 228, such as a cathode ray tube display, a flat panel display, such as a liquid crystal display (LCD), light-emitting diode (LED) display, a plasma display, or any other type of monitor. The computing sub-system 230 of the workstation 226 may be configured to calculate physiological parameters and to show information from the monitor 214 and from other medical monitoring devices or systems (not shown) on the display 228. For example, the workstation 226 may be configured to display an estimate of a patient's blood oxygen saturation generated by the monitor 214 (referred to as an SpO₂ measurement), pulse rate information from the monitor 214 and blood pressure from a blood pressure monitor (not shown) on the display 228.

The monitor 214 may be communicatively coupled to the workstation 226 via a cable 232 and/or 234 that is coupled to a sensor input port or a digital communications port, respectively and/or may communicate wirelessly with the workstation 226. Additionally, the monitor 214 and/or workstation 226 may be coupled to a network to enable the sharing of information with servers or other workstations. The monitor 214 may be powered by a battery or by a conventional power source such as a wall outlet.

The PPG system 210 may also include a fluid delivery device 236 that is configured to deliver fluid to a patient. The fluid delivery device 236 may be an intravenous line, an infusion pump, any other suitable fluid delivery device, or any combination thereof that is configured to deliver fluid to a patient. The fluid delivered to a patient may be saline, plasma, blood, water, any other fluid suitable for delivery to a patient, or any combination thereof. The fluid delivery device 236 may be configured to adjust the quantity or concentration of fluid delivered to a patient.

The fluid delivery device 236 may be communicatively coupled to the monitor 214 via a cable 237 that is coupled to a digital communications port or may communicate wirelessly with the workstation 226. Alternatively, or additionally, the fluid delivery device 236 may be communicatively coupled to the workstation 226 via a cable 238 that is coupled to a digital communications port or may communicate wirelessly with the workstation 226. Alternatively or additionally, the fluid delivery device 236 may be communicatively coupled to one or more other aspects of a fluid responsiveness determination system, such as a fluid responsiveness analysis module or ventilator unit.

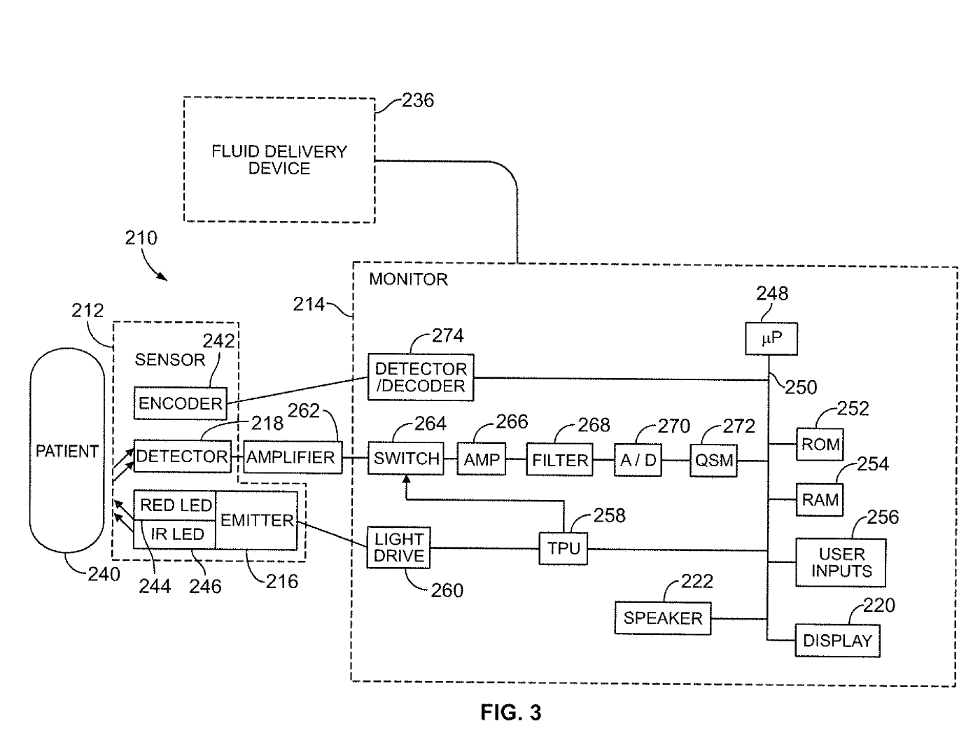

FIG. 3 illustrates a simplified block diagram of the PPG system 210, according to an embodiment. When the PPG system 210 is a pulse oximetry system, the emitter 216 may be configured to emit at least two wavelengths of light (for example, red and infrared) into tissue 240 of a patient. Accordingly, the emitter 216 may include a red light-emitting light source such as a red light-emitting diode (LED) 244 and an infrared light-emitting light source such as an infrared LED 246 for emitting light into the tissue 240 at the wavelengths used to calculate the patient's physiological parameters. For example, the red wavelength may be between about 600 nm and about 700 nm, and the infrared wavelength may be between about 800 nm and about 1000 nm. In embodiments where a sensor array is used in place of single sensor, each sensor may be configured to emit a single wavelength. For example, a first sensor may emit a red light while a second sensor may emit an infrared light.

As discussed above, the PPG system 210 is described in terms of a pulse oximetry system. However, the PPG system 210 may be various other types of systems. For example, the PPG system 210 may be configured to emit more or less than two wavelengths of light into the tissue 240 of the patient. Further, the PPG system 210 may be configured to emit wavelengths of light other than red and infrared into the tissue 240. As used herein, the term “light” may refer to energy produced by radiative sources and may include one or more of ultrasound, radio, microwave, millimeter wave, infrared, visible, ultraviolet, gamma ray or X-ray electromagnetic radiation. The light may also include any wavelength within the radio, microwave, infrared, visible, ultraviolet, or X-ray spectra, and that any suitable wavelength of electromagnetic radiation may be used with the system 210. The detector 218 may be configured to be specifically sensitive to the chosen targeted energy spectrum of the emitter 216.

The detector 218 may be configured to detect the intensity of light at the red and infrared wavelengths. Alternatively, each sensor in the array may be configured to detect an intensity of a single wavelength. In operation, light may enter the detector 218 after passing through the tissue 240. The detector 218 may convert the intensity of the received light into an electrical signal. The light intensity may be directly related to the absorbance and/or reflectance of light in the tissue 240. For example, when more light at a certain wavelength is absorbed or reflected, less light of that wavelength is received from the tissue by the detector 218. After converting the received light to an electrical signal, the detector 218 may send the signal to the monitor 214, which calculates physiological parameters based on the absorption of the red and infrared wavelengths in the tissue 240.

in an embodiment, an encoder 242 may store information about the sensor 212, such as sensor type (for example, whether the sensor is intended for placement on a forehead or digit) and the wavelengths of light emitted by the emitter 216. The stored information may be used by the monitor 214 to select appropriate algorithms, lookup tables and/or calibration coefficients stored in the monitor 214 for calculating physiological parameters of a patient. The encoder 242 may store or otherwise contain information specific to a patient, such as, for example, the patient's age, weight, and diagnosis. The information may allow the monitor 214 to determine, for example, patient-specific threshold ranges related to the patient's physiological parameter measurements, and to enable or disable additional physiological parameter algorithms. The encoder 242 may, for instance, be a coded resistor that stores values corresponding to the type of sensor 212 or the types of each sensor in the sensor array, the wavelengths of light emitted by emitter 216 on each sensor of the sensor array, and/or the patient's characteristics. Optionally, the encoder 242 may include a memory in which one or more of the following may be stored for communication to the monitor 214: the type of the sensor 212, the wavelengths of light emitted by emitter 216, the particular wavelength each sensor in the sensor array is monitoring, a signal threshold for each sensor in the sensor array, any other suitable information, or any combination thereof.

Signals from the detector 218 and the encoder 242 may be transmitted to the monitor 214. The monitor 214 may include a general-purpose control unit, such as a microprocessor 248 connected to an internal bus 250. The microprocessor 248 may be configured to execute software, which may include an operating system and one or more applications, as part of performing the functions described herein. A read-only memory (ROM) 252, a random access memory (RAM) 254, user inputs 256, the display 220, and the speaker 222 may also be operatively connected to the bus 250.

The RAM 254 and the ROM 252 are illustrated by way of example, and not limitation. Any suitable computer-readable media may be used in the system for data storage. Computer-readable media are configured to store information that may be interpreted by the microprocessor 248. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

The monitor 214 may also include a time processing unit (TPU) 258 configured to provide timing control signals to a light drive circuitry 260, which may control when the emitter 216 is illuminated and multiplexed timing for the red LED 244 and the infrared LED 246. The TPU 458 may also control the gating-in of signals from the detector 218 through an amplifier 262 and a switching circuit 264. The signals are sampled at the proper time, depending upon which light source is illuminated. The received signal from the detector 218 may be passed through an amplifier 266, a low pass filter 268, and an analog-to-digital converter 270. The digital data may then be stored in a queued serial module (QSM) 272 (or buffer) for later downloading to RAM 254 as QSM 272 fills up. In an embodiment, there may be multiple separate parallel paths having amplifier 266, filter 268, and A/D converter 270 for multiple light wavelengths or spectra received.

The microprocessor 248 may be configured to determine the patient's physiological parameters, such as SpO₂ and pulse rate, using various algorithms and/or look-up tables based on the value(s) of the received signals and/or data corresponding to the light received by the detector 218. The signals corresponding to information about a patient, and regarding the intensity of light emanating from the tissue 240 over time, may be transmitted from the encoder 242 to a decoder 274. The transmitted signals may include, for example, encoded information relating to patient characteristics. The decoder 274 may translate the signals to enable the microprocessor 248 to determine the thresholds based on algorithms or look-up tables stored in the ROM 252. The user inputs 256 may be used to enter information about the patient, such as age, weight, height, diagnosis, medications, treatments, and so forth. The display 220 may show a list of values that may generally apply to the patient, such as, for example, age ranges or medication families, which the user may select using the user inputs 256.

The fluid delivery device 236 may be communicatively coupled to the monitor 214. The microprocessor 248 may determine the patient's physiological parameters, such as a change or level of fluid responsiveness, and display the parameters on the display 220. In an embodiment, the parameters determined by the microprocessor 248 or otherwise by the monitor 214 may be used to adjust the fluid delivered to the patient via fluid delivery device 236.

As noted, the PPG system 210 may be a pulse oximetry system. A pulse oximeter is a medical device that may determine oxygen saturation of blood. The pulse oximeter may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of a patient. Pulse oximeters typically measure and display various blood flow characteristics including, but not limited to, the oxygen saturation of hemoglobin in arterial blood.

A pulse oximeter may include a light sensor, similar to the sensor 212, that is placed at a site on a patient, typically a fingertip, toe, forehead or earlobe, or in the case of a neonate, across a foot. The pulse oximeter may pass light using a light source through blood perfused tissue and photoelectrically sense the absorption of light in the tissue. For example, the pulse oximeter may measure the intensity of light that is received at the light sensor as a function of time. A signal representing light intensity versus time or a mathematical manipulation of this signal (for example, a scaled version thereof, a log taken thereof, a scaled version of a log taken thereof, and/or the like) may be referred to as a PPG signal. In addition, the term “PPG signal,” as used herein, may also refer to an absorption signal (for example, representing the amount of light absorbed by the tissue) or any suitable mathematical manipulation thereof. The light intensity or the amount of light absorbed may then be used to calculate the amount of the blood constituent (for example, oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with lower oxygen saturation. By comparing the intensities of two wavelengths at different points in the pulse cycle, it is possible to estimate the blood oxygen saturation of hemoglobin in arterial blood.

The PPG system 210 and pulse oximetry are further described in United States Patent Application Publication No. 2012/0053433, entitled “System and Method to Determine SpO₂ Variability and Additional Physiological Parameters to Detect Patient Status,” United States Patent Application Publication No. 2010/0324827, entitled “Fluid Responsiveness Measure,” and United States Patent Application Publication No. 2009/0326353, entitled “Processing and Detecting Baseline Changes in Signals,” all of which are hereby incorporated by reference in their entireties.

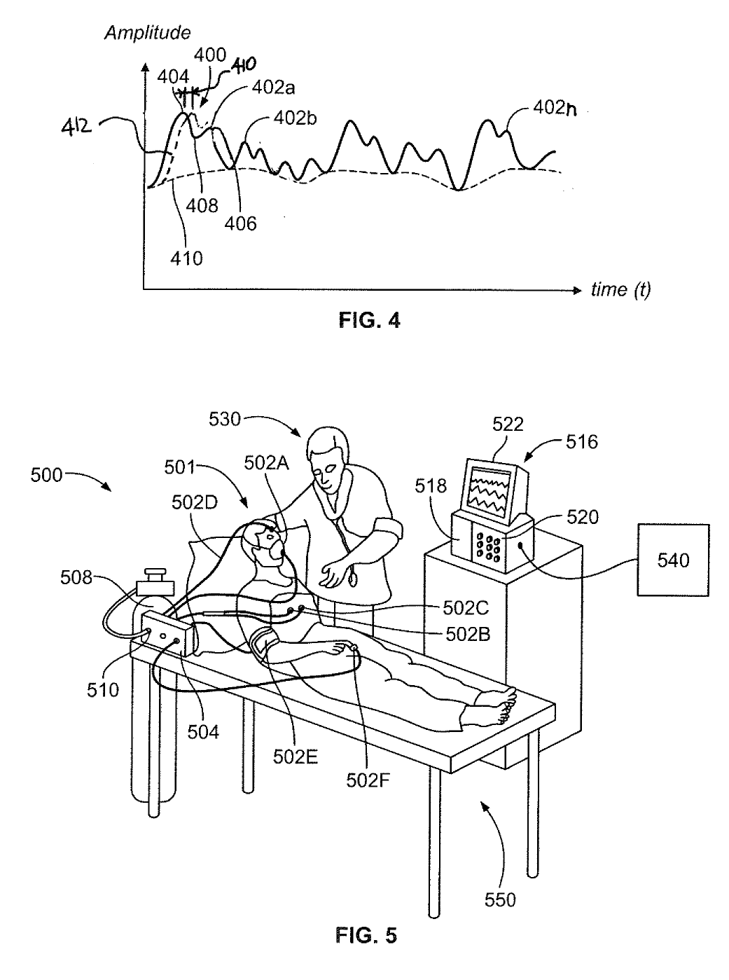

FIG. 4 illustrates a PPG signal 400 over time, according to an embodiment. The PPG signal 400 is an example of a physiological signal. However, embodiments may be used in relation to various other physiological signals, such as a respiratory signal (e.g. a respiratory waveform as discussed above). Certain general principles discussed below in connection with the PPG signal 400 may also apply to other physiological signals. The PPG signal 400 may be determined, formed, and displayed as a waveform by the monitor 214 (shown in FIG. 2) that receives signal data from the PPG sensor 212 (shown in FIG. 2). For example, the monitor 214 may receive signals from the PPG sensor 212 positioned on a finger of a patient. The monitor 214 processes the received signals, and displays the resulting PPG signal 400 on the display 228 (shown in FIG. 2).

The PPG signal 400 may include a plurality of pulses 402a-402n over a predetermined time period. The time period may be a fixed time period, or the time period may be variable. Moreover, the time period may be a rolling time period, such as a 5 second rolling timeframe.

Each pulse 402a-402n may represent a single heartbeat and may include a pulse-transmitted or primary peak 404 separated from a pulse-reflected or trailing peak 406 by a dichrotic notch 408. The primary peak 404 represents a pressure wave generated from the heart to the point of detection, such as in a finger where the PPG sensor 212 (shown in FIG. 2) is positioned. The trailing peak 406 represents a pressure wave that is reflected from the location proximate where the PPG sensor 212 is positioned back toward the heart. One or more features of the PPG signal 400, such as one or more trailing peaks 406 and one or more primary peaks 404, may be used to identify a portion of a PPG signal corresponding to a physiological cycle. Similarly, a signal derived from the PPG signal 400 (e.g. a derivative or integral of the PPG signal 400) may have features, such as one or more peaks, that may be correlated to a physiological cycle. By correlating a feature (e.g. a peak) of the PPG signal 400 (or a signal derived from the PPG signal) with a corresponding feature of another signal and adjusting the PPG signal or the additional signal so that the corresponding features align, the PPG signal and the additional signal may be synchronized.

FIG. 5 provides a perspective view of a multi-parameter monitoring system 500 in accordance with various embodiments. The system 500 includes examples of a respiratory detection module 140 and one or more circulatory detection modules 140, as shown and described with respect to FIG. 1. In FIG. 5, a plurality of patient interfaces are shown positioned proximate to a patient 501, and a plurality of physiological parameters may be collected and/or determined using the multi-parameter monitoring system. One or more of the measured or determined parameters obtained with the use of the monitoring system 500 may be used in determining fluid responsiveness of a patient.

The plurality of patient interfaces may include one or more samplers, sensors, guides, collectors, and the like that may by adapted to sample, sense, collect, or the like a physiological parameter or parameters related to the patient. For example, in the illustrated embodiment, the system 500 includes patient interfaces 502a-502f. The patient interface 502a includes a breath sampler, for example a cannula, adapted to sample exhaled breath of the patient. The patient interfaces 502b-c include heart related sensors, for example electrodes configured to sense a wave associated with the heart. The patient interface 502d includes a sensor configured to sense a brain activity. The patient interface 502e includes a blood pressure related sensor, for example, a non-invasive blood pressure cuff. The patient interface 502f includes a sensor configured to be positioned proximate to an extremity of a patient to sense a circulatory characteristic, for example a pulse oximeter configured to provide PPG information. Other patient interfaces and sensing devices may be employed additionally or alternatively in alternate embodiments. For example, an arterial line catheter may be employed in alternate embodiments.

Some or all of the patient interfaces 502a-502f may be connected to a platform unit 504. The connection between the various patient interfaces and the platform unit 504 may be completely or partially wireless and/or tubeless and may involve the use of appropriate transmitter-receiver interfaces adapted to wireless and/or tubeless connections between the patient interface(s) and the platform unit 504. Alternatively or additionally, one or more of the connections between the various patient interfaces and the platform unit 504 may include a physical connection, such as by wire, cable, tube, or the like. The connection between a given patient interface and the platform unit 504 may be used for the transfer of information or data and/or physical samples (e.g. a sample of exhaled breath). The platform unit may be placed in close proximity to the patient 501, for example at or near a patient bed 550. Further, the platform unit 504 may be portable.

The platform unit 504 may in turn include a variety of constituent components, such as one or more sensors configured to sense parameters of samples acquired via one or more of the various patient interfaces. The platform unit 504 may also include a control center that is user accessible and/or configured to operate automatically. The platform unit 504 may also include adapters configured for connection to various additional devices, power sources, and the like. As one example, the system may include an adapter 510 configured to connect to an oxygen supply, such as a portable tank 508, or as another example, to a central supply, that may be provided to a patient in need. Further still, the platform unit 504 may include or have associated therewith one or more pumps, for example for inflation of a blood pressure cuff, or, as another example, for use in connection with a CO₂ sensor.

The platform unit 504 may further include a communication unit configured to send and receive information (e.g. via a wireless route) between the platform unit 504 and a remote main detection analyzing unit 516 and/or one or more sensors or patient interfaces. The main detection analyzing unit 516 may include several subunits, including, for example, a processor subunit 518 adapted to process or analyze information received form the platform unit 504. The processor subunit 518 may include any applicable hardware and software, and may further include a user interface 520. The user interface 520 is configured to allow the user (e.g. practitioner 530) to control operating parameters and other parameters of the monitoring system 500. In the illustrated embodiment, the main detection analyzing unit 516 also includes a display 522 configured to visually display various parameters related to the operation of the monitoring system 500 and/or parameters being monitored by the monitoring system 500. The main detection analyzing unit 516 may further include a communication subunit configured to allow communication with other aspects or components of the monitoring system 500.

The monitoring system 500 also includes a fluid responsiveness analysis module 540. For example, the fluid responsiveness analysis module 540 may be an example of the fluid responsiveness analysis module 150 as shown and described with respect to FIG. 1. In the illustrated embodiment, the fluid responsiveness analysis module 540 is depicted as a stand-alone component operably connected to the main detection analyzing unit 516. For example, the fluid responsiveness analysis module 540 may receive information describing one or more measured or determined physiological parameters obtained via the main detection analyzing unit 516. Alternatively or additionally, the fluid responsiveness analysis module 540 may receive physiological information directly from one or more of the various patient interfaces and/or the platform unit 504 of the monitoring system 500. In still other embodiments, the fluid responsiveness analysis module 540 may be integrated within the main detection analyzing unit 516.

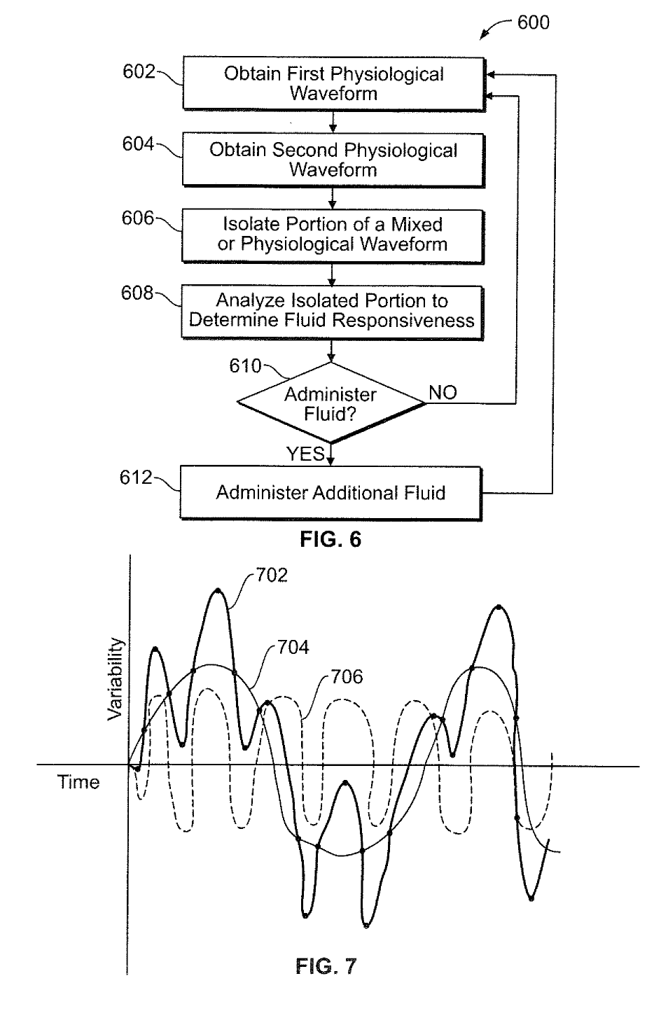

Certain embodiments provide a system and method for determining fluid responsiveness of a patient. In some embodiments, the patient may be ventilated, while in other embodiments, the patient may not be ventilated. For example, FIG. 6 provides a flowchart of a method 600 for determining fluid responsiveness in accordance with various embodiments. In various embodiments, certain steps may be omitted or added, certain steps may be combined, certain steps may be performed simultaneously, or concurrently, certain steps may be split into multiple steps, certain steps may be performed in a different order, or certain steps or series of steps may be re-performed in an iterative fashion. The method 600 may be performed, for example, in association with aspects, components, systems, and/or methods such as those discussed elsewhere herein.

Fluid responsiveness relates to the volume of fluid, such as blood, in the arteries, veins, and vasculature of an individual. In general, fluid responsiveness may include a measurement of the response of stroke volume, the volume of blood passing out of the heart with each heartbeat, to venous return, the volume of blood entering the heart with each heartbeat, caused by the clinical administration of fluid into the vasculature, such as through an intravenous injection. With each heartbeat, a certain amount of blood is pumped out of the heart. The more blood that fills the heart, the more blood the heart can pump out with each heartbeat. If blood volume is too low, the heart may not fully fill with blood. Therefore, the heart may not pump out as much blood with each heartbeat. Consequently, low blood volume may lead to low blood pressure, and organs and tissues may not receive enough blood to optimally and/or properly function. Monitoring fluid responsiveness allows a physician to determine whether additional fluid should be provided to a patient, such as through an intravenous fluid injection. In short, fluid responsiveness represents a prediction of whether or not additional intravenous fluid may improve blood flow within a patient. Fluid responsiveness may be viewed as a response of a heart in relation to overall fluid within a patient.

Fluid responsiveness may be monitored in, for example, critically-ill patients because fluid administration plays an important role in optimizing stroke volume, cardiac output, and oxygen delivery to organs and tissues. However, clinicians need to balance central blood volume depletion and volume overloading. Critically-ill patients are generally at greater risk for volume depletion and severe hypotension is a common life-threatening condition in critically-ill patients. Conversely, administering too much fluid may induce life-threatening adverse effects, such as volume overload, systemic and pulmonary edema, and increased tissue hypoxia. Therefore, obtaining reliable information and parameters that aid clinicians in fluid management decisions may help improve patient outcomes.

An index (e.g. a unitless parameter or percentage) of fluid responsiveness, or index of dynamic preload responsiveness, may be used, to help determine whether the blood flow of a ventilated patient will benefit from additional fluid administration. Such an index may be used to describe a variability corresponding to fluid responsiveness. For example, stroke volume variation (SVV; which may be defined as (SVₘₐₓ−SVₘᵢₙ)/SVₘₑₐₙ over a respiratory cycle) and pulse pressure variation (PPV; which may be defined as automated pulse pressure variations expressed as a percentage) are indices that may currently be obtained using arterial-line pressure waveforms, and the pleth variability index (PVI; which may be defined as (PIₘₐₓ−PIₘᵢₙ)/PIₘₐₓ, where PI=(AC_{IR}/DC_{IR})×100) is an index that may obtained using a PPG. For example, when such an index exceeds a predetermined threshold (e.g. 10%, 15%, or other threshold), additional fluid administration may be indicated. However, use of such indices obtained using current methods may only be supported at higher tidal volumes. For example, SVV obtained by current methods may only be supported for patients who are 100% mechanically ventilated with tidal volumes of more than 8 cc/kg and fixed respiratory rates.

As discussed herein, embodiments of the present disclosure are configured to isolate, on the one hand, the variability in a measured physiological (e.g. circulatory) parameter due to respiration from, on the other hand, variability due to other sources. Such an isolation of variability due to a single source may provide improved accuracy, sensitivity, and/or reliability of determined fluid responsiveness, as well as allow the determination of a fluid responsiveness index for non-ventilated patients and the use of lower tidal volumes in ventilated patients when determining fluid responsiveness. For example, changes in intrathoracic pressure are associated with the breathing process. For example, pressure changes are associated with the movement of the diaphragm to draw air into the lungs and to expel air out of the lungs. The pressure changes in turn affect circulatory parameters, for example as indicated by a blood pressure or a PPG. However, additional variations in blood pressure or PPG are caused by, for example, sources other than respiration-related changes in intrathoracic pressure. For example, differences in ventilator mode, circuit impedance, pressure and flow settings can all affect the size of the waveform variability.

Conceptually speaking, the variability in a waveform may be described by FIG. 7, which illustrates variability in a waveform in accordance with an embodiment. The embodiment shown in FIG. 7 is meant to be illustrative in nature and is not intended to represent any particular signal. The signal 702 represents a sensed signal that modulates from a mean value set at 0 in FIG. 7 over time. The signal 702 may be broken into components 704 and 706, each of which represent a portion of the total signal 702. In the illustrated embodiment, the signal 704 represents a portion of the signal 702 attributable to respiration-related pressure changes, while the signal 706, represented with a dashed line, represents a portion of the signal 702 attributable to all other causes. In some portions, the signals 706 and 704 are additive, and in other portions, the signals 706 and 704 cancel each other out. Due to the confounding effects of the signal portion 706, the variability in the sensed signal 702 differs in many respects to the signal 704. By isolating the change in a waveform due to the change in pressure caused by breathing (either ventilated or spontaneous), a fluid responsiveness attributable to that single cause (e.g. respiration) may be better identified to help provide an improved parameter describing fluid responsiveness.

Returning to FIG. 6, at 602, a first physiological waveform (see, e.g., FIG. 9a and related discussion) is obtained. The first physiological waveform is representative of a physiological activity or process of a patient for whom fluid responsiveness is to be determined. For example, the first physiological waveform may be constructed from first physiological information representative of the physiological activity or process collected or detected by one or more sensors. The first physiological information, for example, may include respiratory information that describes a respiratory output, activity, or process of the patient. The first physiological information may constitute all or a part of the first physiological waveform (e.g. the information may be in the form of a waveform) or the first physiological waveform may be otherwise derived from the first physiological information in either a raw or modified form (e.g. by filtering or normalization).

For example, the respiratory information may correspond to a level of CO₂ within exhaled breath. The respiratory information, in some embodiments, may correspond to a CO₂ concentration, a CO₂ waveform, a change in CO₂ concentration over time, End Tidal CO₂ (Et CO2), or combinations thereof.

In certain embodiments, the respiratory information includes capnography information obtained from a sensor or detector such as a CO₂ sensor. The respiratory information may be obtained via, for example, a detection module such as the respiratory detection module 130 discussed herein. Capnography is a non-invasive monitoring method used to continuously measure the concentration of CO₂ in exhaled breath. Based upon the location of the CO₂ sensor, capnography systems may be divided into two groups referred to as mainstream capnography and sidestream capnography. In mainstream capnography, a CO₂ sensor is located directly between an airway tube and the breathing circuit, and as such, mainstream capnography is primarily limited to use on intubated patients. In sidestream capnography, the CO₂ sensor is remote from the patient and it is located in a main sensing unit. Sidestream capnography may be used with both intubated patients (e.g. by connecting to the intubation tube), as well as non-intubated patient (e.g. by connecting to a mask worn by a patient or the nostrils of the patient). Sidestream capnography may be concurrently performed with other procedures involving the airway of a patient, such as oxygen administration. Sidestream capnography may require a pump or the like to draw a sample of the breath of the patient toward the remote unit for detection, monitoring, analysis, and the like, of CO₂ levels. Typically, sidestream capnography sampling systems are designed taking into consideration that such a pump will create negative pressure being employed.

For example, the respiratory information may include information gathered using a detecting system including a patient interface (e.g. a patient interface similar to patient interfaces 502, discussed herein), a sampling area, and a one or more CO₂ sensors. In some embodiments, the patient interface is configured to be mounted, attached, or associated with a patient, and to collect a sample of the patient's breath. For example, the patient interface may include a mask positioned proximate to a patient's nostrils, or a cannula adapted to collect a sample of exhaled breath from the patient. The sampling area may be located remotely from the patient interface, with the sample of patient's breath drawn toward the sampling area with a pump. At the sampling area, the one or more CO₂ sensors, in conjunction with proximately or remotely located processing equipment, may determine a level or concentration of CO₂ in the sample. As another example, for ventilated patients, CO₂ sensors may be associated with a tube or breathing circuit of a ventilation system.

The respiratory or other first physiological waveform may be constructed directly from readings taken from a sensor or detector to provide a raw waveform, or information obtained from a sensor or detector may be modified or adjusted, for example, by filtering and/or normalizing such information to construct a processed waveform. The sensor or detector may be dedicated for use exclusively in connection with determination of fluid responsiveness, or information from the sensor or detector may be shared with other systems or otherwise used for additional purposes. In embodiments, more than one sensor or detector, or more than one type of sensor or detector may be used to collect the first physiological information (e.g. respiratory information) and/or to obtain the first physiological waveform (e.g. respiratory waveform). Respiratory sensors or sampling units, for example, may be invasively placed (e.g. in conjunction with an endotracheal tube) or non-invasively placed (e.g. in conjunction with a mask or cannula positioned proximate to a patient's nostrils)

In embodiments, the respiratory (or other first physiological) waveform may be obtained directly from a respiratory sensing or detection unit. In other embodiments, the respiratory (or other first physiological) waveform may be obtained directly from a monitoring or processing unit associated with the sensor or detector. In still other embodiments, the respiratory (or other first physiological) waveform may be obtained by a computation using respiratory (or other first physiological) information received from a sensor or a sensor or detector processing unit. For example, a processing unit configured to determine fluid responsiveness may receive respiratory (or other first physiological) information from a sensor and construct the respiratory (or other first physiological) waveform using the received respiratory (or other first physiological) information.

The respiratory (or other first physiological) information and/or respiratory waveform may describe one or more respiratory cycles, or may describe only a portion of one or more respiratory cycles. For example, the respiratory information may include a measurement or indication of Et CO₂.

The first physiological waveform may also be synchronized to another waveform, for example, by adding a time delay to a measured or determined first physiological waveform or to a second physiological waveform to which the first physiological waveform is to be synchronized. In alternate embodiments, different synchronization techniques may be employed. For example, in some embodiments, the first physiological waveform may be synchronized to a PPG waveform as depicted in FIG. 4. The waveforms may be synchronized, for example, by identifying a portion (e.g. a peak such as 404), of the PPG waveform 400 corresponding to a portion of a physiological process such as a point in the respiratory cycle. Then, a portion of the first physiological waveform (for example a physiological or circulatory waveform discussed below) corresponding to the same portion of the physiological process may be identified. A time delay 410 may be determined by identifying the temporal difference between the two points of the respective waveforms, and applying the time delay 410 to the PPG waveform to form a synchronized PPG waveform 412, a portion of which is indicated in dashed line on FIG. 4.

At 604, a second physiological waveform (e.g. a circulatory waveform such as depicted in FIG. 4) is obtained. The second physiological waveform is representative of a physiological activity or process of a patient for whom fluid responsiveness is to be determined. For example, the second physiological waveform may be constructed from physiologic information representative of the physiological activity or process collected or detected by one or more sensors. The second physiological information, for example, may include circulatory information that describes a circulatory activity or process of the patient. The second physiologic information may constitute all or a part of the second physiologic waveform (e.g. the information may be in the form of a waveform) or the second physiologic waveform may be otherwise derived from the second physiologic information in either a raw or modified form (e.g. by filtering or normalization).

For example, the circulatory information may correspond to a level of blood within tissue. In embodiments, the circulatory information includes PPG information obtained from a sensor or detector such as a pulse oximeter positioned at a predetermined position on a patient, for example a fingertip. As another example, the circulatory information may include blood pressure information. For instance, the blood pressure information may correspond to a blood pressure waveform constructed from readings taken with an arterial line catheter. A circulatory or other physiological waveform may be constructed directly from readings taken from a sensor or detector to provide a raw waveform, or information obtained from a sensor or detector may be modified or adjusted, for example, by filtering and/or normalizing such information to construct a processed waveform. The sensor or detector may be dedicated for use exclusively in connection with determination of fluid responsiveness, or information from the sensor or detector may be shared with other systems or otherwise used for additional purposes. In embodiments, more than one sensor or detector, or more than one type of sensor or detector may be used to collect physiological information and to obtain a physiological waveform. Circulatory sensors can be invasively placed (e.g. a catheter) or non-invasively placed (e.g. a pulse oximeter).

Generally speaking, photoplethysmography (PPG) is a non-invasive, optical measurement that may be used to detect changes in blood volume within tissue, such as skin, of an individual. PPG may be used with pulse oximeters, vascular diagnostics, or digital blood pressure detection systems. Typically, a PPG system includes a light source that is used to illuminate skin of a patient, with a photodetector used to measure small variations in light intensity of blood volume proximate the illuminated skin.

In general, a PPG waveform includes an AC physiological component related to cardiac synchronous changes in the blood volume with each heartbeat. The AC component is typically superimposed on a DC baseline that may be related to respiration, sympathetic nervous system activity, and thermoregulation. In some embodiments, a circulatory waveform is obtained by processing an obtained PPG waveform, for example, to remove high frequency artifacts and/or to remove a DC offset. For example, in some embodiments, the PPG waveform may be filtered to remove high frequency offsets. As another example, additionally or alternatively, in some embodiments the PPG waveform may be normalized by a DC value to provide a unit-less modulation depth that is robust to changes in sensor configuration. Thus, a physiological waveform may be obtained by first obtaining a raw waveform and subsequently processing the raw waveform.

As another example, a circulatory waveform may be obtained by measuring arterial line (A-Line) pressure. For example, arterial line pressure may be measured to obtain a waveform by placing a cannula (e.g. an arterial catheter) into an artery. The cannula is operably connected to a fluid filled system which in turn is operably connected to a pressure transducer. Pressure may then be substantially continuously monitored and a waveform of arterial pressure obtained.

In some embodiments, the first and second physiological waveforms may be obtained from sensors or detectors used for additional purposes other than fluid responsiveness determination. For example, the sensors or detectors employed may be part of a multi-parameter monitoring system, such as the system 500 discussed above.

The second physiological waveform (e.g. the circulatory waveform) may be synchronized to the first physiological waveform (e.g. the respiratory waveform as discussed above), for example, by adding a time delay or otherwise aligning the phase of the first and second physiological waveforms. Generally speaking, events in a first waveform (e.g. a respiratory waveform) are identified and tied to events in a second waveform (e.g. a circulatory waveform), and one or both of the first and second waveforms are adjusted so that the corresponding portions of the first and second waveform align, or so that the first and second waveform are in phase with each other. The events may be identified, for example, by identifying peaks or zeros in the waveforms themselves or in derivatives of the waveforms.

For example, the end of expiration may be identified in each of the waveforms. The end of expiration may be identified in the respiratory waveform, and a time delay for the respiratory waveform or the circulatory waveform may be applied so that the portion of the respiratory waveform corresponding to the end of expiration is aligned with a feature of a PPG waveform also corresponding to the end of expiration. In alternate embodiments, a different event may be used, or more than one type of event may be used to align two waveforms or place two waveforms in phase with each other.

In some embodiments, the method 600 may be performed on a non-ventilated patient. In other embodiments, the method 600 may be performed on a ventilated patient. In some embodiments with ventilated patients, obtaining the first physiological waveform 602 and obtaining the second physiological waveform 604 may be performed without varying the ventilator from a predetermined desired treatment operation mode. For example, a predetermined desired treatment operation mode, including settings for one or more of pressure, flow, or volume, may be selected based on desired ventilation for the patient, without regard to the determination of fluid responsiveness. The first and second physiological waveforms may then be obtained without deviating from the predetermined desired treatment operation mode. Thus, a patient's ventilation may be unaltered during fluid responsiveness determination.

In contrast, certain known systems require that a patient's ventilation be manipulated or controlled in a way that deviates from a desired treatment setting, for example, by a series of mechanically controlled breaths, for example, 3. These known systems suffer from a drawback of requiring deviation from a desired treatment setting to obtain a fluid responsiveness index, as well as provide generally limited amounts of time from which to determine fluid responsiveness. Certain embodiments of the present disclosure are configured to allow a patient's ventilation to remain at a predetermined treatment setting without any deviation required for determining fluid responsiveness based on ventilation, thereby avoiding deviation from a predetermined treatment setting as well as allowing for longer sample times, for example about a minute, during which information may be gathered to be used for determining fluid responsiveness. In still other embodiments, the ventilation may be varied from a predetermined treatment setting during data acquisition for determining fluid responsiveness.

In some embodiments, one or both of the first physiological information or the second physiological information may be obtained substantially continuously, for example, in the form of time based measurements at very small intervals, or, as another example, in the form of a wave provided by a sensor or a processing unit associated with the sensor. In other embodiments, one or both of the first physiological information or the second physiological information may be obtained at discrete intervals, for example at a predetermined portion or portions corresponding to a physiological cycle, such as a respiratory cycle. For example, information may be obtained at the end of expiration. A waveform may then be constructed describing a variance over time of a measured or determined parameter at the predetermined portion or portions corresponding to the respiratory cycle.

At 606, a portion of the obtained first and second physiological information and/or a waveform derived from the obtained information is isolated to separate a variability due to respiration from other variabilities in the physiological waveform. Embodiments provide for removal of all or a portion of non-respiratory induced variabilities for improved sensitivity and accuracy of fluid responsiveness variability determinations.

In some embodiments, a “lock-in” technique may be employed to isolate a variation of a waveform that is synchronous with a respiratory cycle. For example, a respiratory waveform (for example, a waveform describing a respiratory output of a patient) may be multiplied by a physiological waveform (for example a PPG waveform, which may be either raw or processed, obtained by a sensor positioned proximate to a patient's finger) to provide a mixed waveform. As also discussed above, the respiratory waveform and the physiological waveform may be synchronized before the two waveforms are multiplied. For example, a time delay may be applied to the respiratory waveform or the physiological waveform to align the waveforms based on corresponding portions of a physiological cycle, such as a breathing cycle. As another example, a lock-in amplifier having an autophase setting may be employed to synchronize the waveforms.

Next, a low pass filter may then be applied to the mixed waveform. (See, e.g., FIGS. 9a and 9b and related discussion). The low pass filter, for example, is selected to have a cut-off frequency lower than a respiration rate associated with the respiration of the patient. Thus, a respiratory responsiveness waveform may be obtained by multiplying the respiratory waveform by the physiological waveform to obtain a mixed waveform, and subsequently applying a low pass filter to the mixed waveform. The respiratory responsiveness waveform corresponds to an isolated variability due to the ventilator cycle, with all or a portion of other contributions to variability filtered and discarded. Next, in some embodiments, the respiratory responsiveness waveform may be normalized. For example, the respiratory responsiveness waveform may be normalized by the amplitude of the respiratory waveform. In embodiments, isolating variability due solely or predominately to respiration allows for improved accuracy, reliability, and predictiveness of fluid responsiveness and/or fluid responsiveness determinations at lower tidal volumes and/or without manipulation of ventilator output from a desired treatment mode of operation and/or without use of a ventilator.

At 608, the resulting respiratory responsiveness waveform is analyzed to determine fluid responsiveness. The respiratory responsiveness waveform analyzed may be, for example, the waveform resulting from the above described application of the low pass filter, or as another example, the waveform resulting from the above described normalization after application of the low pass filter. The respiratory responsiveness waveform may be analyzed, for example, to identify a unitless variability index (expressed as, for example, a fraction, a decimal number, or percentage) describing the respiratory responsiveness. For example, the respiratory responsiveness waveform variability index may be described by (RRₘₐₓ−RRₘᵢₙ)/RRₘₑₐₙ, where RR is the amplitude of the respiratory responsiveness, RRₘₐₓ is the maximum amplitude of the respiratory responsiveness waveform during a predetermined interval, RRₘᵢₙ is the minimum amplitude of the respiratory responsiveness waveform, and RRₘₑₐₙ is the mean amplitude of the respiratory responsiveness waveform. In other embodiments, other measures, indications, or expressions of variability in the respiratory responsiveness waveform may be utilized.

The resulting variability index of the respiratory responsiveness waveform, in some embodiments, may be used directly to determine whether additional fluid administration is appropriate for a given patient. For example, based on clinical studies, a threshold (or thresholds) may be established, with fluid administration appropriate (or a given quantity of fluid administration appropriate) if the threshold is met or exceeded. In some other embodiments, the resulting variability index of the respiratory responsiveness waveform may be used to identify a corresponding value of a previously recognized fluid responsiveness index, such as stroke volume variability (SVV). For example, in a clinical study, the SVV may be concurrently determined using conventional techniques and the variability index of the respiratory responsiveness waveform may be determined using, for example, techniques discussed herein, across a population of patients. By a calibration process, a correlation between the SVV and the variability index of the respiratory responsiveness waveform may be identified. The correlation may be described, for example, by a mathematical function, or as another example, may be described in a look-up table correlating two variability indices. In still other embodiments, a description of the respiratory responsiveness waveform may be calibrated or correlated to an established variability index directly, with, for example, a function or transform determined through clinical studies correlating the respiratory responsiveness waveform and one or more established indices, such as SVV.

In some embodiments, the resulting variability index of the respiratory responsiveness waveform may be adjusted by correction factors for various demographics of patients and/or types of equipment, such as ventilators. The various computations or determinations discussed herein may be performed, for example, by a fluid responsiveness monitoring unit having a processing capability. The fluid responsiveness monitoring unit may, responsive to the determination of a fluid responsiveness index, provide a displayed indication to a practitioner. The displayed indication may include an identification of a determined fluid responsiveness index and/or a recommendation of a fluid administration activity. For example, using the determined fluid responsiveness index (and, in some embodiments, using patient information, for example, identifying a demographic group to which a patient belongs), the fluid responsiveness monitoring unit may develop a recommendation (e.g. “fluid administration not required” or “additional fluid administration indicated”) and/or may display one or more fluid responsiveness variability indices to provide information to a practitioner who will decide if additional fluid administration is performed. The fluid responsiveness monitor in some embodiments is configured as a stand-alone device that may be operably connected, for example, to a main detection processing unit or monitor and/or a ventilator and/or various sensing or detecting devices. In other embodiments, the fluid responsiveness monitor is incorporated into or otherwise associated with, for example, a main detection processing unit or monitor.

At 610, it is determined whether or not fluid is to be administered, using the determined fluid responsiveness. For example, as discussed above, a decision on whether or not to administer additional fluid may be based at least in part on whether or not a threshold of a determined fluid responsiveness index is met or exceeded.

For example, if the threshold is exceeded and it is determined to administer additional fluid, the method proceeds to 612 where additional fluid is administered. The method may then return to 602 to begin a subsequent determination if, at some point after the administration of additional fluid, still further additional fluid administration may be appropriate. If the threshold is not exceeded and it is determined not to administer additional fluid, then the method, for example, may return to 602 for ongoing monitoring to determine if fluid administration becomes appropriate at a later time.

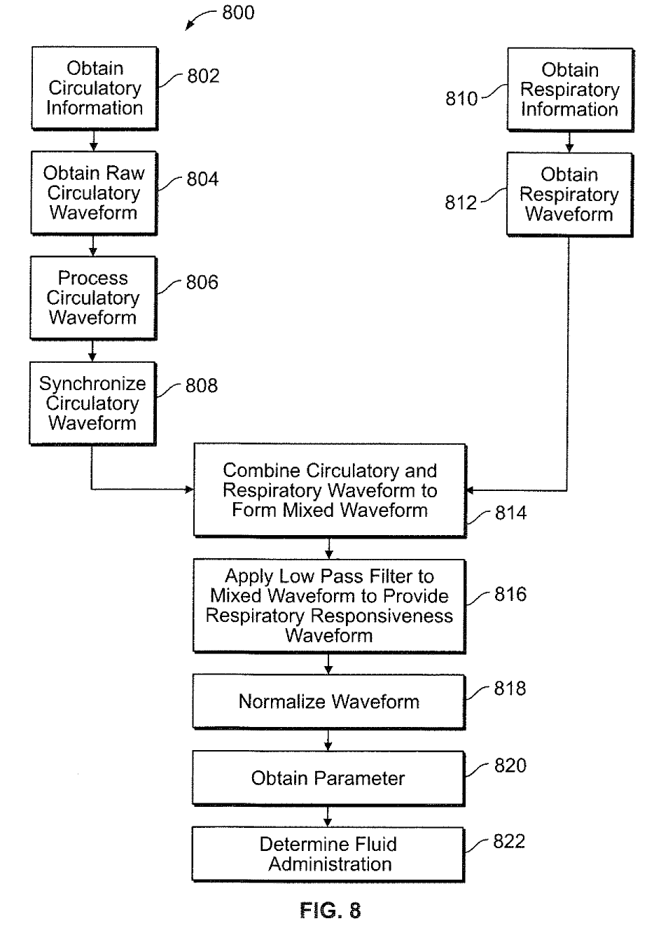

FIG. 8 illustrates a flowchart of a method 800 for determining fluid responsiveness in accordance with various embodiments. In various embodiments, certain steps may be omitted or added, certain steps may be combined, certain steps may be performed simultaneously, or concurrently, certain steps may be split into multiple steps, certain steps may be performed in a different order, or certain steps or series of steps may be re-performed in an iterative fashion. The method 800 may be performed, for example, in association: with aspects, components, systems, and/or methods such as those discussed elsewhere herein.

At 802, physiological information is obtained. For example, the physiological information may include circulatory information describing a circulatory function of a patient. For example, the circulatory information may include information regarding a PPG or a blood pressure, for example a blood pressure measured by a transducer associated with an arterial line catheter. The physiological information may be collected at discrete intervals, or may be collected substantially continuously. In some embodiments, the physiological information includes PPG information, for example obtained with a pulse oximeter located proximate to a patient's finger.

At 804, a raw physiological waveform is obtained. In some embodiments, the raw physiological waveform is a PPG waveform that may be described as W(t). In other embodiments, for example, the raw physiological waveform may describe an arterial pressure. In various embodiments, the raw physiological waveform may be obtained in various ways. For example, the raw physiological waveform may be obtained directly from a sensor. As another example, the raw physiological waveform may be obtained, by a processing unit configured to determine fluid responsiveness, from a separate processing unit associated with a sensor obtaining the physiological information. As still another example, the raw physiological waveform may be constructed at a processing unit configured to determine fluid responsiveness (or a separate processing unit) using information (such as information recorded at discrete intervals) from a sensor or a processing unit associated with a sensor.

At 806, the physiological (e.g. circulatory) waveform is processed. The physiological waveform may be processed, for example, to remove noise or other artifacts, to normalize the physiological waveform, and/or to remove or isolate portions of the physiological waveform for later use. In some embodiments, a PPG waveform is processed by passing the PPG waveform through a bandpass filter and normalizing to remove a DC offset present in the raw PPG waveform due to, for example, respiration, sympathetic nervous system activity, and thermoregulation. The bandpass filter, for example, may define a band from about 0.05 Hz to about 5 Hz. In some embodiments, the raw physiological waveform may be processed at a detection processor associated with the sensor or detector that obtains the raw physiological data. Additionally or alternatively, the raw physiological waveform may be processed at a processing unit, for example, a monitor, configured to determine fluid responsiveness using, among other things, the physiological waveform.

At 808, the physiological waveform is synchronized. For example, the physiological waveform may be synchronized to a respiratory waveform. Generally speaking, the waveforms may be synchronized by identifying portions of each waveform corresponding to a given portion of a physiological cycle, such as a respiratory cycle, and aligning the identified portions of the waveforms. For example, a time delay may be applied to one waveform to synchronize with another. In some embodiments, the time delay may be a generally constant delay added to a function describing a waveform, while in other embodiments, the time delay may vary from cycle to cycle. In the depicted embodiment, the physiological waveform is synchronized to the respiratory waveform by adding a time delay, so that the physiological waveform may be considered as W(t+d), where t is a time and d is a delay added to the time. In alternate embodiments, a time delay may instead by added to an additional physiological waveform to synchronize the additional physiological waveform to the physiological waveform. In alternate embodiments, other techniques of synchronizing or aligning the phase of the waveforms may be employed.

At 810, additional physiological information, for example respiratory information, is obtained. In embodiments, the respiratory information is obtained substantially concurrently with the circulatory information. Alternatively or additionally, the respiratory and circulatory information may be collected and identified with a time stamp or other indicator for use in associating the two waveforms subsequently. The respiratory information may be obtained substantially continuously. Alternatively or additionally, the respiratory information may be obtained at discrete intervals.

At 812, a respiratory waveform is obtained. In some embodiments, the respiratory waveform may be obtained by a fluid responsiveness processing unit that receives a waveform corresponding to a respiratory output of a patient that has been obtained by a sensor or detector. In some embodiments, the respiratory waveform is obtained by constructing a waveform (e.g. the respiratory waveform is constructed by the fluid responsiveness processing unit) using data points received from a sensor or detector. The data points may be collected by the sensor or detector substantially continuously or at discrete time intervals a predetermined time apart or, as another example, corresponding to a portion or portions of a respiratory cycle.

In some embodiments, the respiratory waveform may be obtained, by a fluid responsiveness monitor or processing unit configured to determine fluid responsiveness, by receiving the respiratory waveform from a sensing or detection unit or module that constructs the respiratory waveform using information collected by the sensing or detection unit or module. In other embodiments, the fluid responsiveness monitor or processing unit may obtain the respiratory waveform by constructing the respiratory waveform using information provided by a sensor.

At 814, the circulatory waveform (e.g. (W(t+d)) and the respiratory waveform (e.g. R(t), where R is a function describing respiratory output of a patient) are combined to form a mixed waveform (see, e.g., FIG. 9a and related discussion). In some embodiments, the physiological waveform and the respiratory waveform are multiplied to form the mixed waveform. For example, the mixed waveform “M” may be described as M=R(t)*W(t+d), where d is a time delay applied to the physiological waveform to synchronize the physiological waveform to the respiratory waveform. In alternate embodiments, the time delay may be applied to the respiratory waveform, while in still other embodiments, a different synchronization or phase alignment technique may be employed, such as use of an autophase setting of a lock-in amplifier. Different weightings or coefficients may also be employed in other embodiments. In the depicted embodiment, the multiplication of the respiratory waveform and the circulatory waveform may be performed to help identify and isolate variations in the circulatory waveform induced by respiration from variations caused by other sources.

For example, in the illustrated embodiment, at 816, a low pass filter is applied to remove portions of the mixed waveform that do not correspond to variations induced by respiration. By multiplying the circulatory waveform and the respiratory waveform to form a mixed waveform, and then applying a low pass filter to the mixed waveform to form a respiratory responsiveness waveform, portions of the mixed waveform that do not correspond to respiration induced behavior may be removed, and portions of the mixed waveform attributable to respiration-induced variations may be entirely or partially isolated in the respiratory responsiveness waveform. Thus, in embodiments, such a respiratory responsiveness waveform may provide a more specific representation of the variation due to respiration alone, which in turn may provide improved accuracy and reliability of fluid responsiveness determinations.

FIGS. 9a and 9b illustrate the forming of a mixed waveform and the application of a low pass filter in accordance with an embodiment. Two waveforms may be combined to form the mixed waveform. For example, in the illustrated embodiment, a respiratory waveform 904 (depicted as a generally sinusoidal waveform for clarity of understanding) and a physiological waveform 906 (shown as a dashed line for clarity) are multiplied to form a mixed waveform 902. For example, the physiological waveform may be a PPG waveform. (See, e.g., FIG. 4 and related discussion.) The particular shapes of the waveforms in FIGS. 9a and 9b are intended for clarity of illustration and may vary in practice. In FIG. 9b, the mixed waveform 902 is depicted as a spectrum 910 in a frequency domain. A cut-off frequency 912 is depicted. A low-pass filter having a cut-off frequency of 912 may be applied to the mixed waveform 902 to produce a respiratory responsiveness waveform 920 (represented as a spectrum 922 in the frequency domain in FIG. 9b).

At 818, the respiratory responsiveness waveform is normalized. In some embodiments, the respiratory responsiveness waveform is normalized by the amplitude of the respiratory waveform. For example, normalizing the respiratory responsiveness waveform by the amplitude of the respiratory waveform may quantify the effect of respiration on the waveform variation obtained by the multiplication and filtering (which may be referred to as lock-in detection) discussed above.

At 820, a respiratory responsiveness parameter is obtained using the respiratory responsiveness waveform. For example, the fluid responsiveness parameter may be a unitless parameter (e.g. a percentage) describing the variability of the respiratory responsiveness waveform obtained at 816 and/or 818 above. For example, in some embodiments, a variability of the respiratory responsiveness waveform (referred to herein as a respiratory responsiveness waveform variability index) may be described as (RRₘₐₓ−RRₘₘ)/RRₘₑₐₙ, where RRₘₐₓ corresponds to the maximum amplitude of the respiratory responsiveness waveform, RRₘᵢₙ corresponds to the minimum amplitude of the respiratory responsiveness waveform, and RRₘₑₐₙ corresponds to the mean amplitude of the respiratory responsiveness waveform. In alternate embodiments, other descriptions of the variability of the respiratory responsiveness waveform may be employed.

Further still, additionally or alternatively, in some embodiments, the respiratory responsiveness waveform may be used to obtain a conventionally known fluid responsiveness index, such as SVV. This may be done in one step, using information from the respiratory responsiveness waveform to directly compute the SVV. For example, clinical studies may be used to determine a relationship between the respiratory responsiveness waveform or components or aspects thereof with SVV. Such a relationship, for example, may described by an experimentally derived formulaic relationship. As another example, a conventional fluid responsiveness index, such as SVV, may be obtained in a multi-step process. For instance, the respiratory responsiveness waveform may be analyzed to determine a variability of the respiratory responsiveness waveform, for example as discussed in the preceding paragraph. The respiratory responsiveness waveform variability index may then be converted to a conventionally known or familiar index, such as SVV. The conversion may be accomplished by a formula obtained during a calibration of the respiratory responsiveness waveform variability index to SVV performed during clinical studies. As another example, a lookup table correlating the respiratory responsiveness waveform variability index to SVV may be obtained by a calibration process in clinical studies and utilized to convert the respiratory responsiveness waveform variability index to SVV.

At 822, it is determined if additional fluid administration is appropriate. Such a determined may be made using, for example, the respiratory responsiveness waveform variability index. For example, a threshold or thresholds at which fluid administration is recommended based on the respiratory responsiveness waveform variability index may be determined in clinical studies. As another example, the determination may be made based on a conventional index, such as SVV, with the SVV determined using the respiratory responsiveness waveform or respiratory responsiveness waveform variability index as discussed above. For example, a fluid responsiveness monitor or processing unit that has determined one or more fluid responsiveness parameters (e.g. the respiratory responsiveness waveform variability index, SVV, PVI, or PPV) may display the determined parameter and/or a recommendation for fluid administration based on a predetermined criterion (e.g. a threshold). A practitioner may then determine whether additional fluid administration is appropriate, and administer additional fluid if appropriate.

The method 800 may be performed in an iterative or ongoing fashion. For example, a determined fluid responsiveness index may be substantially continuously displayed, and an alarm or other signal may be activated or otherwise communicated if a threshold is crossed that indicates additional fluid administration is appropriate. In some embodiments, a fluid responsiveness may be determined periodically (e.g. every minute or other predetermined time period) using information collected during the previous minute or other time period) or may be determined on a rolling basis.

Thus, embodiments of the present disclosure provide for the isolation of respiration variability (e.g. variation caused by respiration) in a waveform from other variability (e.g. variation caused by one or more other sources of potential variability), thereby allowing for a more controlled study and determination of fluid responsiveness. For example, embodiments provide systems and methods that are configured to more accurately determine a fluid responsiveness index or indices. Further still, embodiments provide systems and methods configured to determine a fluid responsiveness index for non-ventilated patients.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer.”

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. For example, a module or system may include a computer processor, controller, or other logic-based device that performs operations based on instructions stored on a tangible and non-transitory computer readable storage medium, such as a computer memory. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A system for determining fluid responsiveness of a patient, the system comprising:
a respiratory detection module configured to detect respiratory information representative of respiration of the patient;
a circulatory detection module configured to detect circulatory information representative of circulation of the patient; and
a fluid responsiveness analysis module configured to
obtain a respiratory waveform based at least in part on the respiratory information;
obtain a circulatory waveform based at least in part on the circulatory information;
combine the respiratory waveform and the circulatory waveform to provide a mixed waveform; and
isolate a portion of the mixed waveform to identify a respiratory responsiveness waveform representative of an effect of the respiration of the patient on the mixed waveform.

2. The system of claim 1, wherein the fluid responsiveness analysis module is further configured to determine a fluid responsiveness parameter representative of fluid responsiveness of the patient using the respiratory responsiveness waveform.

3. The system of claim 1, wherein the fluid responsiveness analysis module is further configured to combine the respiratory waveform and the circulatory waveform by multiplication.

4. The system of claim 1, wherein the circulatory detection module comprises a pulse oximetry sensor configured to provide photoplethysmographic information representative of a photopleythsmographic waveform of the ventilated patient.

5. The system of claim 1, wherein the system is configured to be operably connected to a non-ventilated patient, wherein the fluid responsiveness parameter may be determined without the patient being operably connected to a ventilator.

6. The system of claim 1, wherein the respiratory detection module includes a CO₂ sensor, and the respiratory information corresponds to a level of CO₂ in exhaled breath.

7. A method for determining fluid responsiveness of a patient, the method comprising:
obtaining a respiratory waveform representative of a respiratory output of a patient, the respiratory waveform based on information obtained from a respiratory detection module;
obtaining a circulatory waveform representative of circulation of the patient, the circulatory waveform based on information provided by a circulatory detection module;
combining the respiratory waveform and the circulatory waveform to provide a mixed waveform; and
isolating, at a processing module, a portion of the mixed waveform to provide a respiratory responsiveness waveform representative of an effect of respiration of the patient on the mixed waveform.

8. The method of claim 7 further comprising determining, at the processing module, a fluid responsiveness parameter representative of fluid responsiveness of the patient using the respiratory responsiveness waveform.

9. The method of claim 7, wherein combining the respiratory waveform and the circulatory waveform comprises multiplying the respiratory waveform and the circulatory waveform.

10. The method of claim 7, further comprising normalizing the respiratory responsiveness waveform by an amplitude of the respiratory waveform.

11. The method of claim 7, wherein the obtaining the respiratory waveform and the obtaining the circulatory waveform are performed without the patient being operably connected to a ventilator.

12. The method of claim 7, wherein the respiratory waveform corresponds to a level of CO₂ in a breath sample of the patient.

13. The method of claim 7, wherein the patient is ventilated, and the obtaining the respiratory waveform and the circulatory waveform are performed without varying operation of a ventilator from a desired treatment operation mode, wherein the desired treatment operation mode is determined without respect to the determining of the fluid responsiveness parameter.

14. A tangible and non-transitory computer readable medium comprising one or more computer software modules configured to direct a processor to:
obtain a respiratory waveform representative of a respiratory output of a patient, the respiratory waveform based on information obtained from a respiratory detection module;
obtain a circulatory waveform representative of the circulation of the patient, the circulatory waveform based on information provided by a circulatory detection module;
combine the respiratory waveform and the circulatory waveform to provide a mixed waveform; and
isolate a portion of the mixed waveform to provide a respiratory responsiveness waveform representative of an effect of respiration on the mixed waveform.

15. The computer readable medium of claim 14, wherein the computer readable medium is further configured to direct the processor to determine a fluid responsiveness parameter representative of fluid responsiveness of the patient using the respiratory responsiveness waveform.

16. The computer readable medium of claim 14, wherein the computer readable medium is further configured to direct the processor to combine the respiratory waveform and the circulatory waveform by multiplication.

17. The computer readable medium of claim 14, wherein the computer readable medium is further configured to direct the processor to normalize the respiratory responsiveness waveform by an amplitude of the respiratory waveform.

18. The computer readable medium of claim 14, wherein the respiratory waveform and the circulatory waveform are obtained without the patient being operably connected to a ventilator.

19. The computer readable medium in accordance of claim 14, wherein the respiratory waveform corresponds to a level of CO₂ in a breath sample of the patient.

20. The computer readable medium of claim 14, wherein the computer readable medium is further configured to direct the processor to, when the patient is ventilated, obtain the respiratory waveform and the circulatory waveform without varying operation of the ventilator from a desired treatment operation mode, wherein the desired treatment operation mode is determined without respect to the determining of the fluid responsiveness parameter.
